(19) United States
(12) Patent Application Publication
(10) Pub. No.: US 2004/0138446 A1
(43) Pub. Date: Jul. 15, 2004

(54) Biotin derivatives, methods for making same and uses thereof as vectors

(76) Inventors: Abdelakim Kharrat, Montgiscard (FR); Khalil Bouayadi, Toulouse (FR); Jean-Charles Faye, Montesquieu-Volvestre (FR); Emmanuelle Fourcade, Poitiers (FR); Rudi Baron, Durham, NC (US); Daniel Tovar, Villeneuve Tolosane (FR); Casimir Blonski, Ramonville (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/432,383

(22) PCT Filed: Nov. 21, 2001
(86) PCT No.: PCT/FR01/03669

(30) Foreign Application Priority Data
Nov. 22, 2000 (FR) 0015049

(51) Int. Cl. C07D 235/02; C07J 43/00; G01N 33/53

(52) U.S. Cl. 540/107; 548/303.7; 435/7.5

(57) ABSTRACT

The invention concerns biotin derivatives of general formula (I), wherein: R₁ represents a specific compound; X₁ represents a carbonaceous chain, preferably of 1 to 10 carbon atoms, substituted or not, optionally comprising one or several carbonyl or oxycarbonyl groups, and/or a —NH— group, in particular a chain of formula —HN(CH₂)ₙ—O—CO wherein n is an integer between 1 and 5 and optionally comprising one or several ether, amide and/or amino functions; Y represents a hydrocarbon chain of 1 to 5 carbon atoms, substituted or not; R₂ represents a hydrogen atom, or a protecting group, in particular R₂ represents an alkyl group of 1 to 5 carbon atoms. The invention is useful as vectors, in particular for implementing methods for detecting interactions between biological compounds, and in pharmaceutical compositions.

The invention relates to biotin derivatives, and to uses thereof as vectors, in particular in the context of implementing methods for detecting interactions between biological compounds, as well as in the pharmaceutical compositions.

Small molecule-large molecule interactions have an important role in biological processes. They include all ligand-receptor, ligand-transport protein, hormone-receptor, enzyme-substrate, enzyme-inhibitor inter-actions, etc.

The demonstration thereof opens the door to understanding the mechanism of action of physiological molecules, and to the definition of new molecules which can be used in pharmacology.

The development, in yeast, of the “two-hybrid” method (Yang et al., Science, 257, 680-682, 1992) has made it possible to study protein-protein or peptide-protein interactions. This system uses the Gal4 protein, which consists of two independent functional domains: a DNA-binding domain and a transactivating domain. The complex formed by these two domains acts on a promoter of the chimeric Gall-LacZ fusion gene, “reporter gene” of the interaction, and thus induces μ-galactosidase biosynthesis. When the domains are separated, the transcription does not take place. Thus, the DNA-binding domain of Gal4 can be fused to a protein X, and the transcription transactivating region can be fused to a protein Y. If X and Y have the ability to bind to one another, the proximity of the two domains allows activation of the transcription of the Gall-LacZ gene.

However, although this technique is effective for studying peptide-protein interactions in vivo, it does not make it possible to investigate nonpeptide ligands. Another drawback of this technique is the biological tool used: the eukaryotic cells of yeast have a nucleus, where transcription takes place. The proteins of interest must therefore be addressed to the nucleus.

Biotin is a coenzyme, also called vitamin H, synthesized by plants, bacteria and some fungi.

It has the following structure

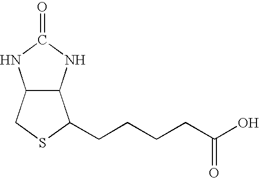

Inside the cells, biotin covalently binds to its carrier proteins via its COOH end, and in particular to a protein referred to as BCCP (for Biotin Carboxylase Carrier Protein), which, in E. coli, is the only protein which accepts binding to biotin (under the action of an intracellular ligase referred to as Bir A).

BCCP is thus biotinylated by formation of an amide bond between the COOH function of biotin (represented above) and the NH₂ group of a lysine residue (located approximately 34 to 35 residues from the carboxy terminal end of the amino acid sequence of BCCP). The protein thus biotinylated has an enzymatic role in metabolic carboxylation/decarboxylation reactions.

The present invention comes from the discovery made by the inventors that biotin (or derivatives) in which the nitrogen atom located at position 1′ (N1′) is substituted with a given compound makes it possible to integrate this compound into target cells, without, however, there being:

firstly, breaking of the bond between said given compound and the biotin,

and, secondly, masking of the site for binding (namely the abovementioned COOH function) of biotin to BCCP.

Thus, the inventors have demonstrated a new method of introducing given compounds which are biotinylated (while at the same time conserving biotin's property of binding to its carrier protein, such as BCCP) into target cells, in particular bacterial cells, advantageously E. coli cells, or yeast, or cells of the human or animal body (in particular CHO cells).

A subject of the invention is the biotin derivatives substituted in the 1′-position, of general formula (I) below

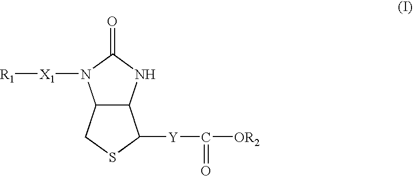

in which

R₁ represents a given compound,

X₁ represents a carbonaceous chain, preferably of 1 to 10 carbon atoms, which may or may not be substituted, comprising, where appropriate, one or more carbonyl or oxycarbonyl groups and/or an —NH— group, in particular a chain of formula

HN—(CH₂)ₙ—O—CO— in which n is an integer from 1 to 5, and comprising, where appropriate, one or more ether, amide and/or amine functions,

Y represents a hydrocarbon-based chain of 1 to 5 carbon atoms, which may or may not be substituted,

R₂ represents a hydrogen atom or a protective group, in particular R₂ represents an alkyl group of 1 to 5 carbon atoms.

A subject of the invention is more particularly the abovementioned biotin derivatives of formula (I) in which X₁ represents a chain of formula —HN—(CH₂)ₙ—O—CO in which n represents 2 or 4, Y represents a hydrocarbon-based chain of 4 carbon atoms, and R₂ is a hydrogen atom or methyl group.

In the preceding and subsequent text, the term “given compound” is intended to mean any chemical molecule of synthetic or natural origin, or any biological molecule of synthetic or natural origin, which may or may not be protein based, in particular any abovementioned molecule which is biologically active and, where appropriate, which can have a pharmacological action of interest in human or animal organisms. It may also be, in a nonlimiting manner, a fluorescent compound, a peptide or an oligonucleotide.

Particularly preferred biotin derivatives of formula (I) are chosen from the following:

the compounds of formula (Ia) below

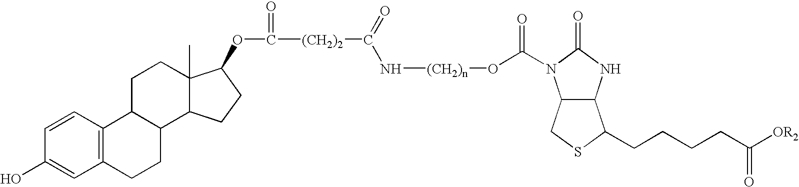

in which:

R₁ represents a radical derived from estradiol hemisuccinate of formula below

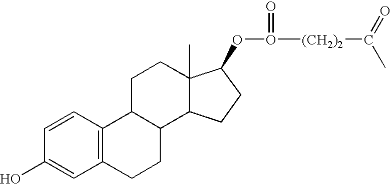

X₁ represents a chain of formula —HN—(CH₂)ₙ—O—CO in which n=2 or 4, Y represents a hydrocarbon-based chain of 4 carbon atoms, and R₂ represents —H or —CH₃,

the compounds of formula (Ib) below

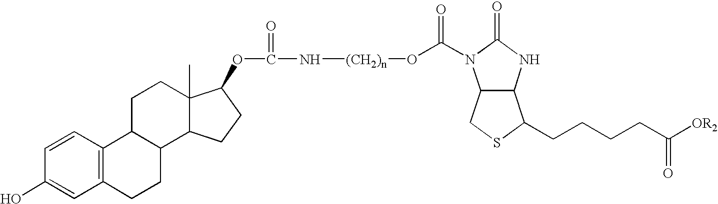

in which

R₁ represents a radical derived from estradiol of formula below

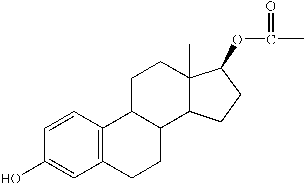

X₁ represents a chain of formula —HN—(CH₂)ₙ—O—CO in which n=2 or 4, Y represents a hydrocarbon-based chain of 4 carbon atoms, and R₂ represents —H or —CH₃,

the compounds of formula (Ic) below

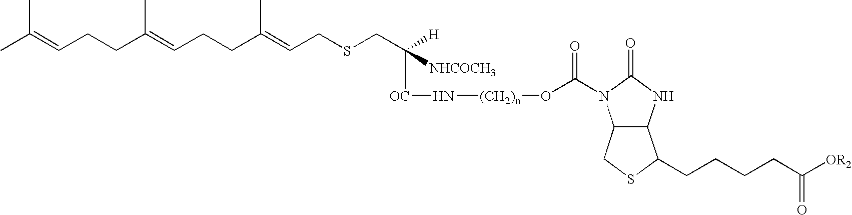

in which

R₁ represents a radical derived from N-acetyl-S-farnesylcysteine of formula below

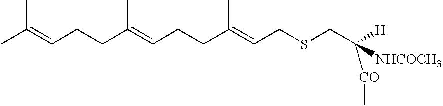

X₁ represents a chain of formula HN—(CH₂)ₙ—O—CO in which n=2 or 4, Y represents a hydrocarbon-based chain of 4 carbon atoms, and R₂ represents —H or —CH₃,

the compounds of formula (Id) below

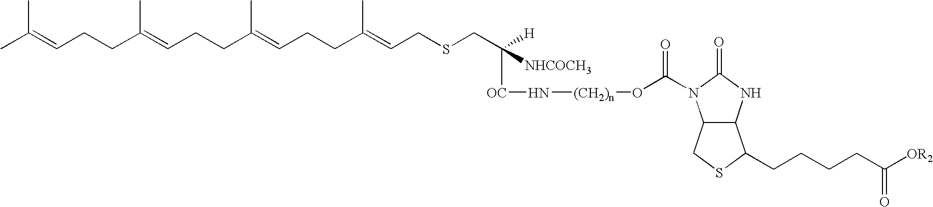

in which

R₁ represents a radical derived from N-acetyl-S-geranylgeranylcysteine of formula below

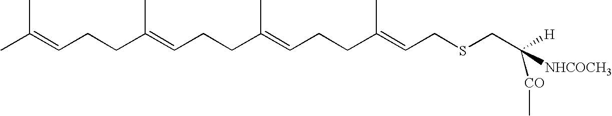

X₁ represents a chain of formula HN—(CH₂)ₙ—O—CO in which n=2 or 4, Y represents a hydrocarbon-based chain of 4 carbon atoms, and R₂ represents —H or —CH₃,

the compounds of formula (Ie) below:

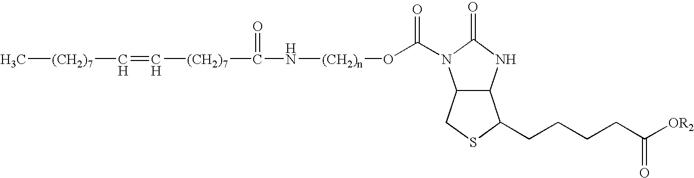

in which

R₁ represents a radical derived from oleic acid of formula below

H₃C—(CH₂)₇—CH═CH—(CH₂)₇—CO—

X₁ represents a chain of formula —HN— (CH₂)ₙ—O—CO— in which n=2 or 4, Y represents a hydrocarbon-based chain of 4 carbon atoms, and R₂ represents —H or —CH₃.

The invention relates even more particularly to the biotin derivatives of formula (I) described above, in which R₂ is a hydrogen atom.

A subject of the invention is also the biotin derivatives of formula (II) described below, and use thereof for preparing abovementioned derivatives of formula (I), by coupling an appropriate function of a given compound (this function being, where appropriate, pre-grafted onto said given compound) and the function R of said derivatives of formula (II), in particular according to the one of the coupling methods described below.

The abovementioned biotin derivatives of general formula (II) correspond to the formula below:

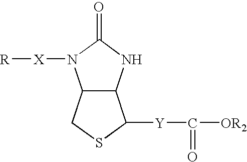

in which

R represents a function —COOH, —NH₂ or —N₃, or a halogen atom,

X represents a carbonaceous chain, preferably of 1 to 10 carbon atoms, which may or may not be substituted, comprising, where appropriate, one or more carbonyl or oxycarbonyl groups, in particular a chain of formula —(CH₂)ₙ—O—CO in which n is an integer of 1 to 5,

Y represents a hydrocarbon-based chain of 1 to 5 carbon atoms, which may or may not be substituted,

R₂ represents a hydrogen atom or a protective group, in particular R₂ represents an alkyl group of 1 to 5 carbon atoms, in particular a methyl group.

A subject of the invention is more particularly the abovementioned biotin derivatives of formula (II) in which R is a halogen atom such as I or Cl, X represents a chain of formula —(CH₂)ₙ—O—CO— in which n represents 2 or 4, Y represents a hydrocarbon-based chain of 4 carbon atoms, and R₂ is a methyl group.

Particularly preferred biotin derivatives of formula (II) are those of formulae below

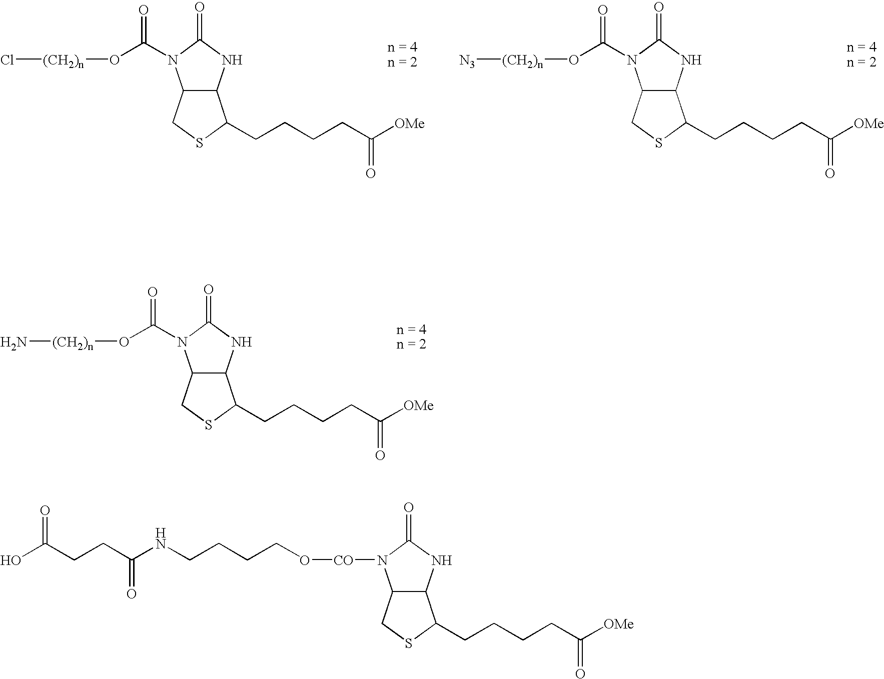

The synthesis of the abovementioned biotin derivatives of formula (I) and (II) is advantageously carried out according to the method comprising the following steps:

treating a biotin derivative of formula below

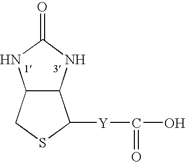

in which Y is as defined above, with a compound of formula R₂—OH, in which R₂ represents a protective group as defined above, in particular a CH₃ group, in the presence of an ion exchange resin, advantageously at approximately 70° C. for approximately 7 hours, which produces the compound of formula below

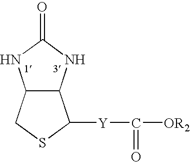

in which Y and R₂ are as defined above,

bringing the ester obtained in the preceding step into contact with a compound of formula R—X—Cl in which R is as defined above, in particular R represents a halogen atom such as Cl or I, and X is as defined above, in particular X represents a chain of formula —(CH₂)ₙ—O—CO— in which n represents 2 or 4, advantageously at approximately 65° C. for 4 days, which leads to the production of the compound of formula (IIa) below

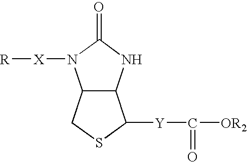

in which X, R and Y are as defined above, and R₂ represents a protective group,

where appropriate, treating the above-mentioned compound of formula (IIa) in which R represents a halogen atom, with NaN₃/NaI, advantageously at approximately 60° C. for 4 days, which leads to the production of the compound of formula (IIb), namely a compound of formula (II) in which R represents N₃, X and Y are as defined above, and R₂ represents a protective group,

where appropriate, hydrogenating the above-mentioned compound of formula (IIb), which leads to the production of the compound of formula (IIc), namely a compound of formula (II) in which R represents NH₂, X and Y are as defined above, and R₂ represents a protective group,

where appropriate, coupling the above-mentioned compounds of formulae (IIa), (IIb) and (IIc) with a given compound, using a coupling reagent, such as BOP/BOH in the presence of triethylamine, which leads to the production of compounds of above-mentioned formula (I), in particular:

coupling β-estradiol 17-hemisuccinate of formula below

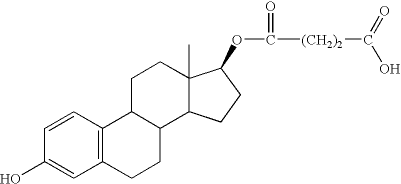

to the compound of formula (IIc) below

in which n represents 2 or 4, which leads to the production of compounds of abovementioned formula (Ia) below:

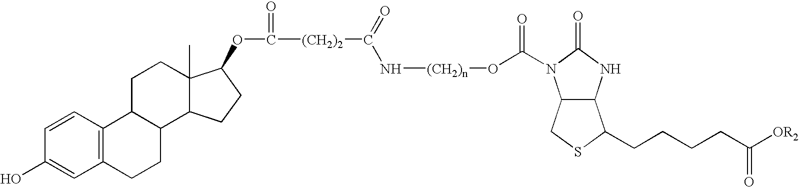

in which n=2 or 4, and R₂═CH₃,

coupling 17-estradiol-17-yl 1,2,2,2-tetrachloroethyl carbonate of formula below

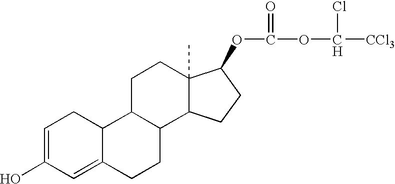

to the compound of abovementioned formula (IIc-1), which leads to the production of compounds of abovementioned formula (Ib) below

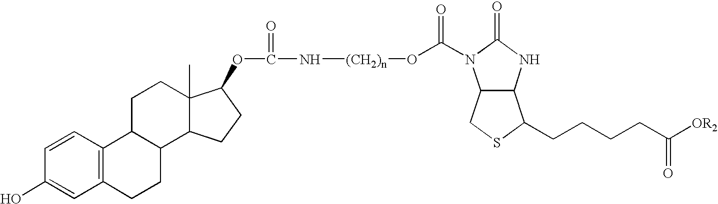

in which n=2 or 4, and R₂═CH₃,

coupling N-acetylfarnesylcysteine of formula below:

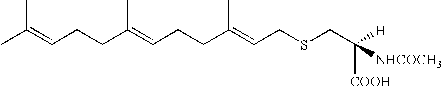

to the abovementioned compound of formula (IIc-1), which leads to the production of compounds of abovementioned formula (Ic) below

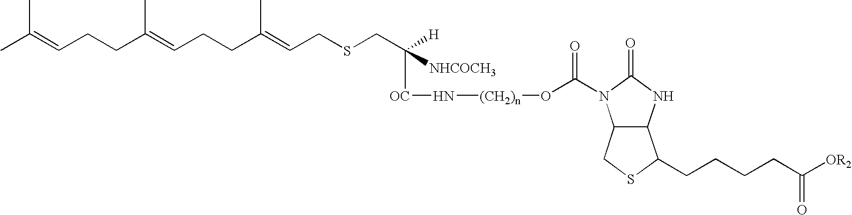

in which n=2 or 4, and R₂═CH₃,

coupling N-acetyl-S-geranylgeranylcysteine of formula below

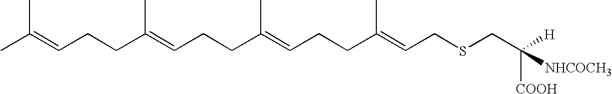

to the compound of abovementioned formula (IIc-1), which leads to the production of compounds of abovementioned formula (Id) below

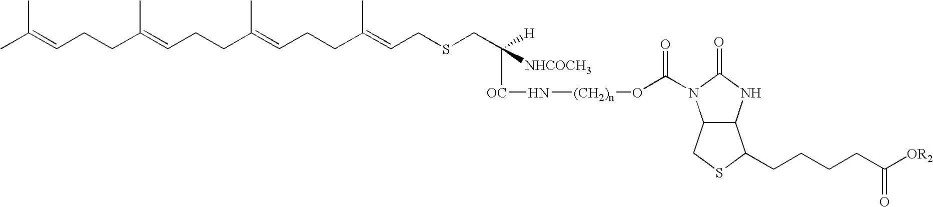

in which n=2 or 4, and R₂═CH₃,

coupling oleic acid of formula below

H₃C—(CH₂)₇—CH═CH—(CH₂)₇— COOH

to the compound of abovementioned formula (IIc), which leads to the production of compounds of above-mentioned formula (Ie) below

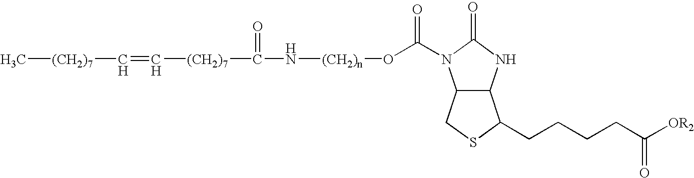

in which n=2 or 4, and R₂═CH₃,

where appropriate, deprotecting the compounds of formulae (IIa), (IIb) and (IIc) and of formula (I), namely of abovementioned formulae (Ia), (Ib), (Ic), (Id) and (Ie), in particular:

by treatment of said compounds with an esterase, such as pig liver esterase (PLE), advantageously at ambient temperature for approximately 10 days, or

by acid catalysis of said compounds, in particular by treatment with sulfuric acid at 65° C. for 24 hours, which leads to the production of compounds of formulae (IIa), (IIb) and (IIc) and of formula (I), namely of abovementioned formulae (Ia), (Ib), (Ic), (Id) and (Ie), in which R₂═H.

The invention relates to the use of biotin derivatives of abovementioned formula (II), as vectors for introducing a given compound into target cells, when said given compound is linked to the radical R of said derivatives of formula (II).

The invention also relates to the use of the biotin analogues of formula (I), as vectors for introducing a given compound into target cells.

The invention relates more particularly to the abovementioned use of biotin derivatives defined above, in the context of implementing a method for detecting proteins or other nonprotein molecules capable of interacting with said given compound, or in the context of preparing medicinal products for introducing a given compound of therapeutic interest into cells of the organism.

Thus, the aim of the invention is to provide novel methods for detecting interactions between biological compounds, making it possible to study the interaction between, firstly, a given protein compound or a nonprotein compound and, secondly, a protein compound or a nonprotein compound tested for its ability to interact with said given compound. The term “nonprotein compound” is intended to mean any compound which cannot be produced by a mechanism of nucleic acid transcription, as opposed to a protein compound which is capable of being produced by such a mechanism.

A subject of the invention is more particularly any method for detecting proteins capable of reacting with the radical R₁ (corresponding to the given compound) of a biotin derivative of formula (I) as defined above, characterized in that it comprises:

bringing said biotin derivative of formula (I) into contact with host cells transformed so as to contain:

a DNA sequence encoding a first fusion protein comprising BCCP and a first revealing protein, and

a DNA sequence encoding a second fusion protein comprising a protein capable of interacting with the abovementioned radical R₁ and a second revealing protein,

the two revealing proteins being chosen in such a way that the bringing together thereof, subsequent to an interaction between the protein capable of interacting with the radical R₁ in said second fusion protein, and the radical R₁ of said biotin derivative, engenders a reaction process the result of which can be detected, this bringing into contact being carried out for a period of time sufficient to allow the transport of said biotin derivative into said host cells, and the binding of said biotin derivative to the BCCP of said first fusion protein,

possibly detecting the result of the reaction process due to the bringing together of the two revealing proteins, then reflecting an interaction between said protein capable of interacting with the radical R₁, and said radical R₁.

The invention is more particularly directed toward the use of the abovementioned method for detecting proteins capable of interacting with a given nonprotein compound, in particular in the context of screening a library of nucleic acids encoding proteins capable of interacting with said given compound.

The various nucleic acids of said library are coupled with the DNA sequence encoding the abovementioned second revealing protein, and the fusion sequences thus obtained, placed under the control of a transcription promoter, are used to transform host cells by means of a suitable vector.

The host cells in which a reaction reflecting an interaction between the protein(s) encoded by one or more abovementioned nucleic acids and the given compound occurs, are isolated and the amino acid sequences of said proteins are analyzed.

A subject of the invention is also any method for detecting protein or nonprotein molecules, also referred to hereinafter as tested molecules, capable of interacting with the radical R₁ of a biotin derivative of formula (I) as described above, characterized in that it comprises

bringing

a first biotin derivative of formula (I) in which R₁ represents the given compound with which a tested molecule is capable of interacting, and

a second biotin derivative of formula (I) in which R₁ represents the tested molecule, into contact with host cells transformed so as to contain:

a DNA sequence encoding a first fusion protein comprising BCCP and a first revealing protein, and

a DNA sequence encoding a second fusion protein comprising BCCP and a second revealing protein, the two revealing proteins being chosen in such a way that the bringing together thereof, subsequent to an interaction between said tested molecule of the second biotin derivative and said radical R₁ of the first biotin derivative, engenders a reaction process the result of which can be detected, this bringing into contact being carried out for a period of time sufficient to allow the transport of the abovementioned first and second biotin derivatives into said host cells, and the binding of said biotin derivatives to the BCCP of said fusion proteins,

possibly detecting the result of the reaction process due to the bringing together of the two revealing proteins, which reflects an interaction between said tested molecule of the second biotin derivative and said radical R₁ of the first biotin derivative.

Advantageously, the screening methods described above are used to detect interactions between said tested proteins or nonprotein molecules, and a given biologically active nonprotein compound, in particular a compound of therapeutic interest.

Preferably, the two revealing proteins used in the abovementioned detection methods are chosen in such a way that:

the first revealing protein corresponds to a DNA-binding domain (such as the GAL4 binding domain described in the article by M. Johnston et al., A model for fungal gene regulatory mechanism the GaI genes of Saccharomyces cerevisiae, Microbiological Reviews, 51, 458-476, 1987), while the second revealing protein corresponds to a domain for activation of the transcription of a reporter gene (such as the GAL4 activating domain which activates transcription of the gene encoding β-galactosidase, in particular β-GalHis3), such that the detection of the product of transcription of the reporter gene corresponds to the detection of an interaction between the given compound and the tested protein or nonprotein molecule, or

the first revealing protein corresponds to a DNA-binding domain (such as the LexA binding domain described in the following articles: B. E. Markham et al., Nucleotide sequence of the lexA gene of Escherichia coli K-12, Nucleic Acids Research, 9, 4149-4160, 1981; P. Oertel-Buchheit et al., Spacing requirements between LexA operator half-sites can be relaxed by fusing the LexA DNA binding domain with some alternative dimerization domains, J Mol Biol., 229, 1-7, 1993), while the second revealing protein corresponds to a dimerization domain (such as the LexA dimerization domain), such that the absence of detection of the product of transcription of a reporter gene (such as the gene encoding β-galactosidase) corresponds to the detection of an interaction between the given compound and the tested protein or nonprotein molecule, or

the first revealing protein corresponds to a first fluorescent protein (such as GFP, for Green Fluorescent Protein, described in the article by A. Crameri et al., Improved Green Fluorescent Protein by Molecular Evolution Using DNA Shuffling, Nature Biotechnology, 14, 315-319, 1996; GFP: Clontech Mutant pGFPuv Bacterial Vector, GenBank Accession #U62636), while the second revealing protein corresponds to a second fluorescent protein (such as BFP, for Blue Fluorescent Protein, Clontech mutant, pBBFP-NI Vector, Catalog # 6069-1), the two fluorescent proteins being chosen in such a way that the bringing together thereof, subsequent to an interaction between said given compound and the tested protein or nonprotein molecule, engenders a process of fluorescence transfer such that the detection of the fluorescence characteristic of the first fluorescent protein at the excitation wavelength of the second fluorescent protein corresponds to the detection of an interaction between the given compound and the tested protein or nonprotein molecule.

Preferably, the host cells used in the context of implementing the abovementioned methods are bacterial cells, advantageously E. coli cells, or are yeast, or else are higher eukaryotic cells.

Advantageously, the compound of formula (I) defined above, and more particularly those of formulae (Ia), (Ib), (Ic), (Id) and (Ie), used in the context of implementing the abovementioned methods, are compounds of formula (I) in which R₂ represents a hydrogen atom.

An aim of the invention is also to provide novel compounds for implementing such detection methods.

In this respect, a subject of the invention is thus any nucleotide sequence encoding a fusion protein comprising BCCP fused to a revealing protein as described above, in particular the BCCP of Escherichia coli.

In particular, a subject of the invention is more particularly:

the nucleotide sequence containing the DNA sequence encoding the GAL4 DNA-binding domain, said DNA sequence being linked to the DNA sequence encoding BCCP,

the nucleotide sequence containing the DNA sequence encoding the GAL4 activating domain, said DNA sequence being linked to the DNA sequence encoding BCCP,

the nucleotide sequence containing the DNA sequence encoding the LexA DNA-binding domain, said DNA sequence being linked to the DNA sequence encoding BCCP,

the nucleotide sequence containing the DNA sequence encoding the LexA dimerization domain, said DNA sequence being linked to the DNA sequence encoding BCCP,

the nucleotide sequence containing the DNA sequence encoding GFP, said DNA sequence being linked to the DNA sequence encoding BCCP,

the nucleotide sequence containing the DNA sequence encoding BFP, said DNA sequence being linked to the DNA sequence encoding BCCP.

A subject of the invention is also a vector, in particular plasmid, containing a nucleotide sequence as defined above.

The invention also relates to the host cells, in particular of E. coli, transformed with a vector defined above.

The subject of the invention is also any fusion protein comprising BCCP fused to a revealing protein as described above.

In this respect, a subject of invention is more particularly:

the protein from fusion between the GAL4 DNA-binding domain and BCCP,

the protein from fusion between the GAL4 activating domain and BCCP,

the protein from fusion between the LexA DNA-binding domain and BCCP,

the protein from fusion between the LexA dimerization domain and BCCP,

the protein from fusion between GFP and BCCP,

the protein from fusion between BFP and BCCP.

The invention also relates to the biotin derivatives of abovementioned formula (I), in which a fusion protein as defined above is linked to the carboxyl function carried by Y.

As regards the nucleotide sequences encoding the fusion proteins described in the above-mentioned methods, the portion of these sequences encoding BCCP can be replaced with a sequence encoding another protein able to be recognized by biotin and to bind to the latter in the cellular host system used.

A subject of the invention is also the sets or kits which can be used to implement a detection method as described above of the invention, comprising

host cells transformed with abovementioned nucleotide sequences encoding fusion proteins described above, and/or

biotin derivatives as described above, and more particularly abovementioned derivatives of formula (II) onto the radical R of which can be grafted the abovementioned given compound or tested molecule.

The subject of the invention is also the biotin derivatives of formula (I), as described above, in which the radical R₁ represents a compound of therapeutic interest.

In this respect, a subject of the invention is more particularly the biotin derivatives of abovementioned formulae (Ia), (Ib), (Ic), (Id) and (Ie).

The invention also relates to the pharmaceutical compositions comprising one or more biotin derivatives of formula (I), as described above, in which the radical R₁ represents a compound of therapeutic interest, in combination with a pharmaceutically acceptable vehicle.

A subject of the invention is also the use of biotin derivatives of the formula (I), as described above, in which the radical R₁ represents a compound of therapeutic interest, for preparing medicinal products for introducing said compound of therapeutic interest into cells of the organism, said medicinal products being intended for the treatment of pathological conditions against which said compound of therapeutic interest is active.

In this respect, a subject of the invention is more particularly the use of compounds of abovementioned formulae (Ia), (Ib), (Ic), (Id) and (Ie), for preparing a medicinal product intended for the treatment of breast cancer, of atheroma, of osteoporosis, or which can be used in the context of cicatrization.

The invention is more particularly directed toward the use of compounds of abovementioned formula (Ic), in particular of the compound (10) described below, as regulators of cell proliferation, in particular for preparing a medicinal product intended for the treatment of atheroma, or which can be used as a cicatrizing agent.

The invention is more particularly directed toward the use of compounds of abovementioned formula (Id) and (Ie), in particular the compounds (20) and (13) described below, as inhibitors of cell proliferation, in particular for preparing a medicinal product intended for the treatment of cancers, such as breast cancer.

The invention also relates to the use of biotinylated compounds consisting of biotin, or of derivatives thereof, in which the nitrogen atom in the 1′-position (N1′) is directly or indirectly linked to a given compound, as vectors for introducing a given compound into target cells.

The term “biotin derivatives” is intended to mean any molecule derived by substitution of one or more atoms of biotin other than N1′, in particular by substitution of the carbonyl group adjacent to the N1′ atom with a —NH— group, or by substitution of the S atom with Se or O, and/or in which the side chain of formula —(CH₂)₄COOH is modified in its length and/or by substitution.

According to one embodiment of the invention, said given compound is directly linked to the N1′ atom via one of its functions capable of reacting with the N1′ atom. Where appropriate, said given compound is modified so as to carry this function capable of reacting with the hydrogen atom carried by N1′.

According to another embodiment of the invention, the N1′ nitrogen atom of biotin, or of derivatives thereof, is substituted with a function capable of forming a covalent bond with a function of said given compound, the latter being, where appropriate, modified so as to carry this other function.

The invention also relates to the use of the abovementioned biotin, or biotin derivatives, in which the N1′ nitrogen atom is directly or indirectly substituted with a given compound, for implementing methods for detecting proteins or other nonprotein molecules capable of interacting with said given compound, and more particularly methods as described above.

A subject of the invention is also the use of the abovementioned biotin, or biotin derivatives, in which the N1′ nitrogen atom is directly or indirectly substituted with a given compound of therapeutic interest, for preparing medicinal products for introducing said compound of therapeutic interest into cells of the organism, said medicinal products being intended for the treatment of pathological conditions against which said compound of therapeutic interest is active.

The invention will be further illustrated using the following detailed description of the methods for preparing biotin derivatives of the invention, and of the construction of vectors and of host cells for implementing a method according to the invention for detecting interactions between nonprotein compounds, and protein or nonprotein compounds.

I) Experimental Section—Synthesis

The compounds used come from Sigma-Aldrich.

d-Biotin methyl ester (2). The d-biotin (1) (10 mmol, 2.44 g) is dissolved in 75 ml of anhydrous methanol. 2.6 g of Amberlite 1R120 ion exchange resin, washed with 2N hydrochloric acid, rinsed with water until neutrality and then dried, beforehand, are added. The solution is brought to reflux (70°) for 7 hours under an inert atmosphere. After returning to ambient temperature, the resin is filtered off and rinsed several times with hot methanol. The filtrate is recovered and the methanol is evaporated off under reduced pressure. 2 g (79.5% yield) of product (2) are obtained in the form of a white powder.

TLC on silica: Rf=0.41 (MeOH:CHCl₃, 5:95).

N-1′-(4-Chloro-1-butoxycarbonyl)-d-biotin Methyl Ester (3).

The d-biotin methyl ester (2) (1 mmol), 258 mg) is dissolved in 5 ml of hot anhydrous chloroform. 18 mmol (3.1 g, 2.46 ml) of 4-chlorobutyl chloroformate are added and the solution is brought to reflux (65° C.) for 4 days. After returning to ambient temperature, the chloroform is evaporated off under reduced pressure and the yellow solution obtained is added dropwise to 250 ml of petroleum ether at 4° C. The mixture is maintained at 4° C. for approximately 15 minutes, until a pale yellow precipitate appears, which is recovered by filtration. This precipitate is solubilized in 1 ml of dichloromethane and purified by flash chromatography on silica (MeOH:CH₂Cl₂, 3:97). 350 mg (89.5% yield) of product (3) are obtained in the form of a thick yellow oil.

TLC on silica: Rf=0.33 (MeOH:CH₂Cl₂, 3:97).

Elemental analysis for C₁₆H₂₅ClN₂O₅S (392.90): calculated C, 48.91; H, 6.41; N, 7.13, O, 20.36. Found C, 48.97; H, 6.46; N, 7.12, O, 20.36.

N-1′-(2-Chloro-1-ethoxycarbonyl)-d-biotin Methyl Ester (3′).

The d-biotin methyl ester (2) (1 mmol, 258 mg) is dissolved in 5 ml of hot anhydrous chloroform. 18 mmol (2.57 g, 1.86 ml) of 2-chloroethyl chloroformate are added and the solution is brought to reflux (65° C.) for 4 days. After returning to ambient temperature, the chloroform is evaporated off under reduced pressure and the yellow solution obtained is added dropwise to 250 ml of petroleum ether at 4° C. The mixture is maintained at 4° C. for approximately 15 minutes, until a pale yellow precipitate appears, which is recovered by filtration. This precipitate is solubilized in 1 ml of dichloromethane and purified by flash chromatography on silica (MeOH:CH₂Cl₂, 3:97). 291 mg (80% yield) of product (3′) are obtained in the form of a thick yellow oil.

TLC on silica: Rf=0.33 (MeOH:CH₂C12, 3:97).

N-1′-(4-Azido-1-ethoxycarbonyl)-d-biotin Methyl Ester (4).

1 mmol (392 mg) of (3) and 0.1 mmol (17 mg) of NaI are dissolved in 8 ml of acetone. The solution is made homogeneous by stirring. 5 mmol (325 mg) of NaN₃ dissolved in 2 ml of water are added. The solution is brought to reflux (60° C.) for 4 days in the dark. After returning to ambient temperature, the acetone is evaporated off under reduced pressure and the water is removed by lyophilization. The yellow oil obtained is taken up in 5 ml of dichloromethane. A white precipitate appears, which is separated from the solution by filtration. The filtrate is recovered and the dichloromethane is evaporated off under reduced pressure. The residue is purified by flash chromatography on silica (MeOH:CH₂C12, 4:96). 379 mg (95% yield) of product (4) are obtained in the form of a yellow oil.

TLC on silica: Rf=0.33 (MeOH:CH₂Cl₂, 3:97).

Elemental analysis for C₁₆H₂₅N₅O₅S (399.47): calculated C, 48.11; H, 6.31; N, 17.53, O 20.03. Found C, 45.03; H, 6.49; N, 17.29.

N-1′-(2-Azido-1-ethoxycarbonyl)-d-biotin Methyl Ester (4′).

1 mmol (364 mg) of (3′) and 0.1 mmol (17 mg) of NaI are dissolved in 8 ml of acetone. The solution is made homogeneous by stirring. 5 mmol (325 mg) of NaN₃ dissolved in 2 ml of water are added. The solution is brought to reflux (60° C.) for 4 days in the dark. After returning to ambient temperature, the acetone is evaporated off under reduced pressure and the water is removed by lyophilization. The yellow oil obtained is taken up in 5 ml of dichloromethane. A white precipitate appears, which is separated from the solution by filtration. The filtrate is recovered and the dichloromethane is evaporated off under reduced pressure. The residue is purified by

Flash chromatography on silica (MeOH:CH₂Cl₂, 4:96). 289 mg (78% yield) of product (4′) are obtained in the form of a yellow oil.

TLC on silica: Rf=0.33 (MeOH:CH₂Cl₂, 3:97).

N-1′-(2-Amino-1-butoxycarbonyl)-d-biotin Methyl Ester (5).

0.75 mmol (300 mg) of (4) are dissolved in 10 ml of ethanol. 150 mg of Pd/C are added, and the solution is placed under an atmosphere of H₂. After stirring for 4 hours at ambient temperature, the solution is filtered through filter paper and the Pd/C is rinsed several times with methanol. The filtrate is recovered and the methanol is evaporated off under reduced pressure. 210 mg (75% yield) of product (5) are obtained in the form of a yellow oil.

TLC on silica: Rf=0 (MeOH:CH₂Cl₂, 3:97).

Hydrochloride of (5): The compound (5) is taken up in 3 ml of water. Hydrochloric acid is added until a pH=4 is obtained. The solution is lyophilized and the hydrochloride is obtained in the form of pale yellow crystals.

Elemental analysis for C₁₆H₂₈ClN₃O₅S (409.93): calculated C, 46.88; H, 6.88; N, 10.25. Found C, 47.05; H, 6.95; N, 10.12.

N-1′-(2-Amino-1-ethoxycarbonyl)-d-biotin Methyl Ester (5′).

0.75 mmol (278 mg) of (4′) are dissolved in 10 ml of ethanol. 150 mg of Pd/C are added, and the solution is placed under an atmosphere of H₂. After stirring for 4 hours at ambient temperature, the solution is filtered through filter paper and the Pd/C is rinsed several times with methanol. The filtrate is recovered and the methanol is evaporated off under reduced pressure. 181 mg (70% yield) of product (5′) are obtained in the form of a yellow oil.

TLC on silica: Rf=O(MeOH:CH₂Cl₂, 3:97).

N-1′-(4-(β-Estradiol 17-(succin-1-ate-4-amido))-1-butoxycarbonyl-d-biotin methyl ester (7)

54 mmol (20 mg) of β-estradiol 17-hemisuccinate (6) are dissolved in 5 ml of anhydrous dimethylformamide. 81 mmol (8.9 μl) of N-methylmorpholine, 108 mmol (47.8 mg) of BOP (benzotriazolyl-N-oxytris(dimethyl-amino)phosphonium hexafluorophosphate) and 108 mmol (14.6 mg) of BOH(N-hydroxybenzotriazole) are added. The solution is placed under an inert atmosphere. 108 mmol (40.3 mg) of N-1′-(4-amino-butoxycarbonyl)-d-biotin methyl ester (5) are dissolved in 4 ml of anhydrous dimethylformamide to which 216 mmol (30.4 μl) of triethylamine have been added. The biotin solution is added to the β-estradiol 17-hemisuccinate solution and the mixture is placed under an inert atmosphere and stirred at ambient temperature for 4 days. The solution becomes dark yellow. The dimethylformamide is evaporated under reduced pressure by formation of an azeotrope with toluene. The residue is solubilized in 10 ml of dichloromethane and the solution obtained is washed four times with water and then once with a saturated aqueous solution of NaCl. Next, the organic phase is dried over MgSO₄, and then purified by flash chromatography on silica (MeOH:CH₂Cl₂, 5:95). Since the purification fraction obtained is not always pure, it is purified by High Performance Liquid Chromatography (C18 column, eluent 95/5 acetonitrile/water, flow rate 1 ml/min, UV detection at 280 nm). 19.2 mg (49% yield) of product (7) are obtained in the form of a yellow oil.

TLC on silica: Rf=0.35 (MeOH:CH₂Cl₂₁ 8:92).

N-1′-(4-(N-Acetyl-S-farnesylcysteinamido)-1-butoxy-carbonyl)-d-biotin Methyl Ester (9)

53.6 mmol (19.7 mg) of N-acetyl-S-farnesylcysteine (8) synthesized according to the method of L. Zhang and P. Casey, The Journal of Biological Chemistry, Vol. 269, No. 23, pp. 15973-15976, 1994) are dissolved in 5 ml of anhydrous dimethylformamide. 80.4 mmol (8.8 μl) of N-methylmorpholine, 107.2 mmol (47.4 mg) of BOP (benzotriazolyl-N-oxytris(dimethylamino)phosphonium hexafluorophosphate) and 107.2 mmol (14.5 mg) of BOH (N-hydroxybenzotriazole) are added. The solution is placed under an inert atmosphere. 107.2 mmol (40 mg) of N-1′-(4-amino-1-butoxycarbonyl)-d-biotin methyl ester (S) are dissolved in 4 μl of anhydrous dimethylformamide to which 214.4 mmol (30 ml) of triethylamine have been added. The biotin solution is added to the N-acetyl-S-farnesylcysteine solution and the mixture is placed under an inert atmosphere and stirred at ambient temperature for 4 days. The solution becomes dark yellow. The dimethylformamide is evaporated under reduced pressure by formation of an azeotrope with toluene. The residue is solubilized in 10 ml of dichloromethane and the solution obtained is washed four times with water and then once with a saturated aqueous solution of NaCl. Next, the organic phase is dried over MgSO₄, and then purified by flash chromatography on silica (MeOH:CH₂Cl₂, 5:95). Since the purification fraction obtained is not always pure, it is purified by High Performance Liquid Chromatography (C18 column, eluent 50/50 acetonitrile/water, flow rate 2 ml/min, UV detection at 220 nm). 17 mg (44% yield) of product (9) are obtained in the form of a yellow oil.

TLC on silica: Rf=0.5 (MeOH:CH₂C12, 8:92).

N-1′-(4-(N-Acetyl-S-farnesylcysteinamido)-1-butoxy-carbonyl)-d-biotin (10)

7 mmol (5 mg) of (9) are solubilized in 500 ml of ethanol. This solution is added to 4.5 ml of a KH₂PO₄/Na₂HPO₄ buffer solution, pH=8, containing 70U of pig liver esterase (1U of enzyme deprotects 1 mmol of substrate per minute at pH 8 and 25° C.). The solution is left to stir at ambient temperature for 3 days, adding 70U of pig liver esterase each day. The reaction mixture is acidified to pH=3 with a 0.1M HCl solution, and then 3 extractions are carried out with 5 ml of ethyl acetate. The organic solution is then dried over MgSO₄ and the ethyl acetate is evaporated off under reduced pressure. 0.8 mg (16% yield, of product (10) is obtained in the form of a yellow oil.

TLC on silica: Rf=0 (MeOH:CH₂Cl₂, 8:92).

N-1′-(4-Oleylamido-1-butoxycarbonyl)-d-biotin Methyl Ester (12).

0.1 mmol (28.25 mg) of oleic acid (11) are dissolved in 5 ml of anhydrous dimethylformamide. 0.15 mmol (16.5 μl) of N-methylmorpholine, 0.2 mmol (88 mg) of BOP (benzotriazolyl-N-oxytris(dimethylamino)phosphonium hexafluorophosphate) and 0.2 mmol (27 mg) of BOH (N-hydroxybenzotriazole) are added. The solution is placed under an inert atmosphere. 0.18 mmol (71 mg) of N-1′-(4-amino-1-butoxycarbonyl)-d-biotin methyl ester (5) are dissolved in 4 μl of anhydrous dimethylform-amide to which 0.4 mmol (56 ml) of triethylamine have been added. The biotin solution is added to the oleic acid solution and the mixture is placed under an inert atmosphere and stirred at ambient temperature for 4 days. The solution becomes dark yellow. The dimethylformamide is evaporated under reduced pressure by formation of an azeotrope with toluene. The residue is solubilized in 10 ml of dichloromethane and the solution obtained is washed 4 times with water and then once with a saturated aqueous solution of NaCl. The organic phase is then dried over MgSO₄. The dichloromethane is evaporated off under reduced pressure and the residue is taken up in 5 ml of diethyl ether. The appearance of a white precipitate is observed, which is filtered through a buchner funnel. The filtrate is recovered, the ether is evaporated off under reduced pressure, and the residue is purified by flash chromatography on silica (MCOH:CH₂Cl₂, 3:97). 35 mg (54% yield) of product (12) are obtained in the form of a yellow oil.

TLC on silica: Rf=0.45 (MeOH:CH₂Cl₂, 8:92).

HRMS (FAB+, MeOH) m/z: 638.41958 (638.42028 calc. for C₃₄Hs₉N₃O₆₅, M+—H)

N-1′-(4-Oleylamido-1-butoxycarbonyl)-d-biotin (13).

Acid hydrolysis. 100 μl of 2N H₂SO₄ are added to a solution of (12) (80 mg, 0.13 mmol) in a mixture of 30 ml of acetonitrile and 10 ml of water. The resulting solution is stirred at 70° C. for 48 hours. The acetonitrile is evaporated off and the aqueous phase is extracted 3 times with 10 ml of dichloromethane. The combined organic phases are washed once with water saturated with NaCl, dried (Na₂SO₄), and concentrated under reduced pressure, so as to obtain 39 mg of (13) in the form of a yellow oil (48% yield).

TLC on silica: Rf=0.25 (MeOH:CH₂Cl₂, 8:92).

HRMS (FAB+MeOH) m/z: 646.38715 (646.38658 calc. for C₃₃H₅₇N₃O₆S MNa⁺)

17 β-Estradiol-17-yl 1,2,2,2-tetrachloroethyl carbonate (15)

0.37 mmol (100 mg) of 17 β-estradiol (14) and 0.37 mmol (30 μl) of anhydrous pyridine are dissolved in 3 ml of anhydrous tetrahydrofuran. A solution containing 0.37 mmol (57 μl) of 1,2,2,2-tetrachloroethyl chloro-formate dissolved in 2 ml of anhydrous tetrahydrofuran is added. A yellow precipitate appears immediately. The solution is stirred at ambient temperature for 16 hours. The precipitate is filtered off through a buchner funnel and rinsed with 2 ml of tetrahydrofuran. The filtrate is recovered and the tetrahydrofuran is evaporated off under reduced pressure. The residue is purified by flash chromatography (MeOH:CH₂C12, 1:99). 25 mg (14.4% yield) of product (15) are obtained in the form of a yellow oil.

TLC on silica: Rf 0.49 (MeOH:CH₂Cl₂, 1:99).

N-1′-(4-(17 β-Estradiol 17-oxyamido)-1-butoxycarbonyl)-d-biotin Methyl Ester (16)

41.5 μmol (20 mg) of 17 β-estradiol-17-yl 1,2,2,2-tetrachloroethyl carbonate (15) and 41.5 μmol (15 mg) of N-1′-(4-amino-1-butoxycarbonyl)-d-biotin methyl ester (5) are dissolved in 6 ml of tetrahydrofuran and 100 μl of water. 41.5 μmol (3.5 μl) of pyridine are added. A precipitate forms immediately. The reaction is left to stir at ambient temperature for 48 hours. The solvent is evaporated off under reduced pressure and the residue is taken up in 5 ml of ethyl acetate. This solution is washed 3 times with 5 ml of water, and then once with 5 ml of water saturated with NaCl. The organic phase is then dried over Na₂SO₄. After evaporation of the ethyl acetate under reduced pressure, the residue is purified by flash chromatography (MeOH:CH₂Cl₂, 3:997). 6 mg (21.5% yield) of product (16) are obtained in the form of a yellow oil.

TLC on silica: Rf 0.30 (MeOH:CH₂Cl₂, 5:95).

N-1′-(4-(17 β-Estradiol 17-(succin-1-ate-4-amido))-1-butoxycarbonyl)-d-biotin (17)

5.5 μmol (40 mg) of N-1′-(4-(17 β-estradiol 17-succin-1-ate-4-amido))-1-butoxycarbonyl)-d-biotin methyl ester (7) are dissolved in 15 ml of an acetonitrile/water mixture (2:1). The pH of the solution is brought to 0.7 by adding concentrated H₂SO₄. The solution is left to stir at 65° C. for 24 hours. It is then neutralized by adding 1N NaOH. The acetonitrile is removed by evaporation under reduced pressure, and the remaining aqueous phase is extracted several times with 5 ml of ethyl acetate. 26 mg (66% yield) of product (17) are obtained in the form of a yellow oil.

TLC on silica: Rf 0.14 (MeOH:CH₂Cl₂₁ 8:92). N-1′-(4-(N-Acetyl-S-geranylgeranylcysteinamido)-1-butoxycarbonyl)-d-biotin methyl ester (19) 92 μmol (40 mg) of N-acetyl-S-geranylgeranylcysteine (18) are dissolved in 5 ml of anhydrous dimethylform-amide. 38 μmol (15.2 μl) of N-methylmorpholine, 92 μmol (40.7 mg) of BOP (benzotriazolyl-N-oxytris(dimethyl-amino)phosphonium hexafluorophosphate) and 92 μmol (12.4 mg) of BOH(N-hydroxybenzotriazole) are added. The solution is placed under an inert atmosphere. 1.84 μmol (68.6 mg) of N-1′-(4-amino-1-butoxycarbonyl)-d-biotin methyl ester (5) are dissolved in 4 μl of anhydrous dimethylformamide to which 368 μmol (51.3 μl) of triethylamine have been added. The biotin solution is added to the N-acetyl-S-geranylgeranylcysteine solution, and the mixture is placed under an inert atmosphere and stirred at ambient temperature for 4 days. The solution becomes dark yellow. The dimethylformamide is evaporated under reduced pressure by formation of an azeotrope with toluene. The residue is solubilized in 10 ml of dichloromethane and the solution obtained is washed 4 times with water and then once with a saturated aqueous solution of NaCl. Next, the organic phase is dried over MgSO₄, and then purified by flash chromatography (MeOH:CH₂Cl₂, 3:97). 44.5 mg (70% yield) of product (19) are obtained in the form of a yellow oil.

TLC on silica: Rf=0.56 (MeOH:CH₂Cl₂, 4:96).

N-1′-(4-(N-Acetyl-S-geranylgeranylcysteinamido)-1-butoxycarbonyl)-d-biotin (20)

50.6 μmol (40 mg) of N-1′-(4-(N-acetyl-S-geranyl-geranylcysteinamido)-1-butoxycarbonyl)-d-biotin methyl ester (19) are dissolved in 15 ml of an acetone/water mixture (2:1). The pH of the solution is brought to 0.7 by adding 2M H₂SO₄. The solution is allowed to stir at ambient temperature for 3 days. It is then neutralized by adding 1N NaOH. The acetone is removed by evaporation under reduced pressure, and the remaining aqueous phase is extracted several times with 5 ml of dichloromethane. 31 mg (90% yield) of product (20) are obtained in the form of a yellow oil.

TLC on silica: Rf 0.30 (MeOH:CH₂Cl₂, 4:96).

N-1′-[(4-n-Butan-4-oic-amido)-1-butoxycarbonyl]-d-biotin methyl ester (21)

A solution of 200 mg (0.54 mmol) of (5) and 108 mg (1.08 mmol) of succinic anhydride in 5 ml of dimethylformamide is stirred at ambient temperature for 24 hours. The dimethylformamide is evaporated off under reduced pressure. 10 ml of dichloromethane are added to the residue and the solution is extracted 3 times with 10 ml of water and washed once with water saturated with NaCl. The organic phase is dried (Na₂SO₄) and concentrated to give 71 mg of N-1′-[(4-N-butan-4-oicamido)-1-butoxycarbonyl]-d-biotin methyl ester in the form of a yellow oil (28% yield).

TLC on silica: Rf=0.73 (CH₂Cl₂:MeOH:acetic acid 85:14:1).

HRMS (FAB+MeOH) m/z: 474.19169 (474.19101 calc. for C₂₀H₃₁N₃O₈S MH+)

The synthesis of the compounds (1) to (21) are summarized in the following scheme.

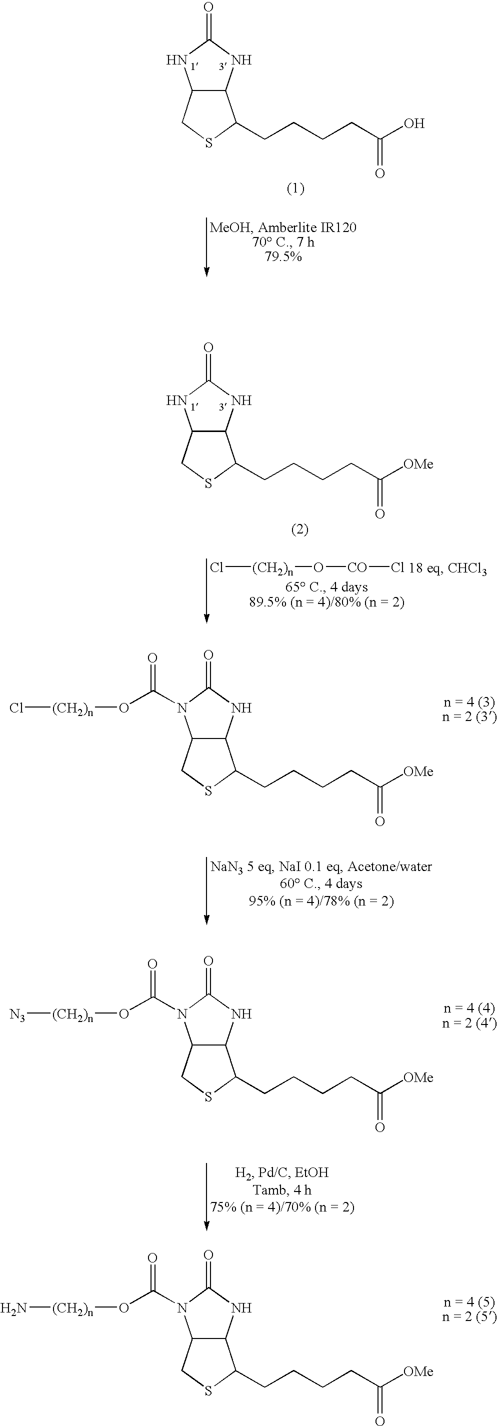

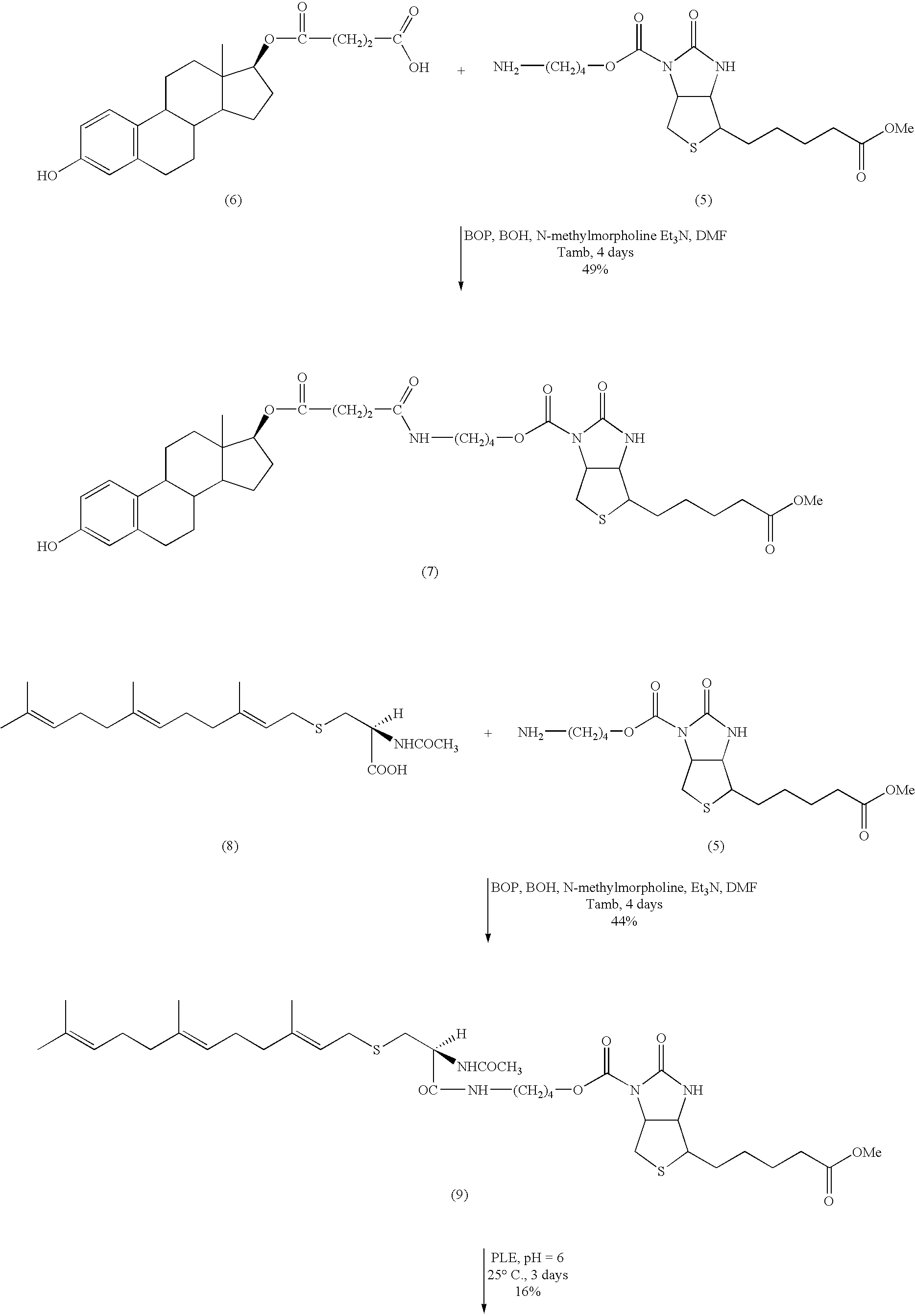

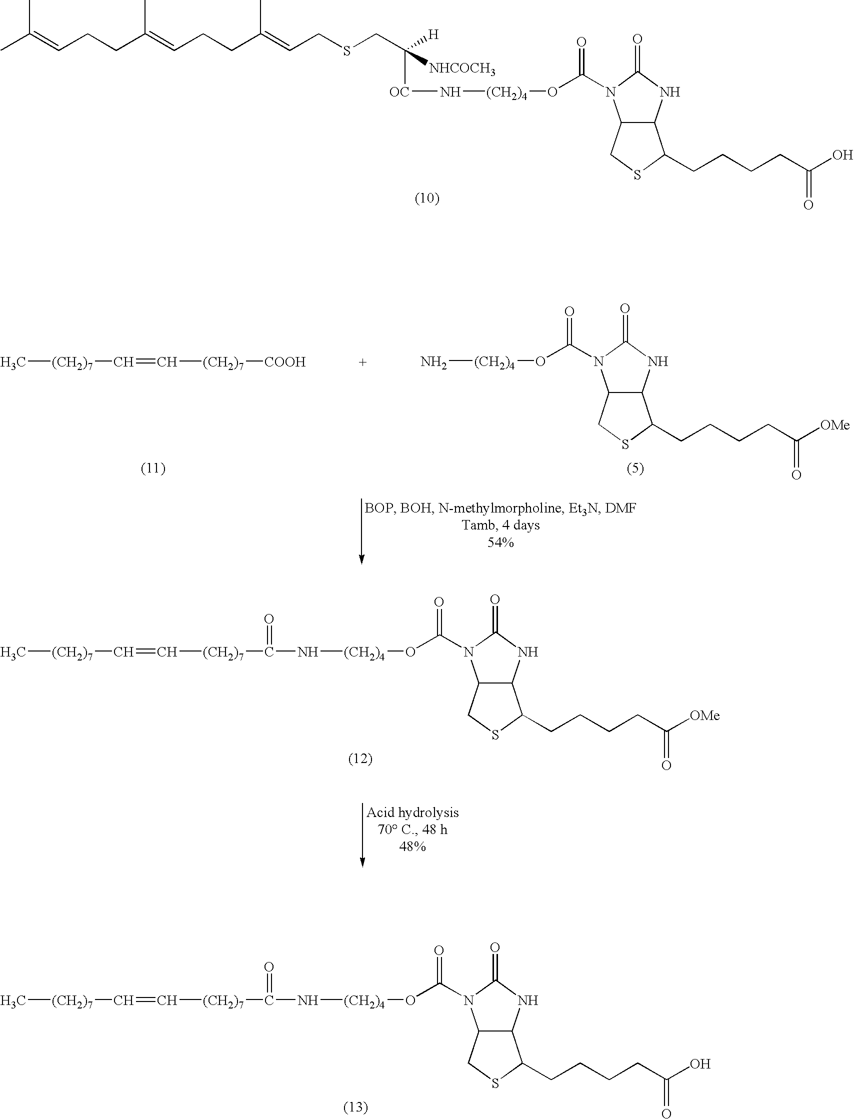

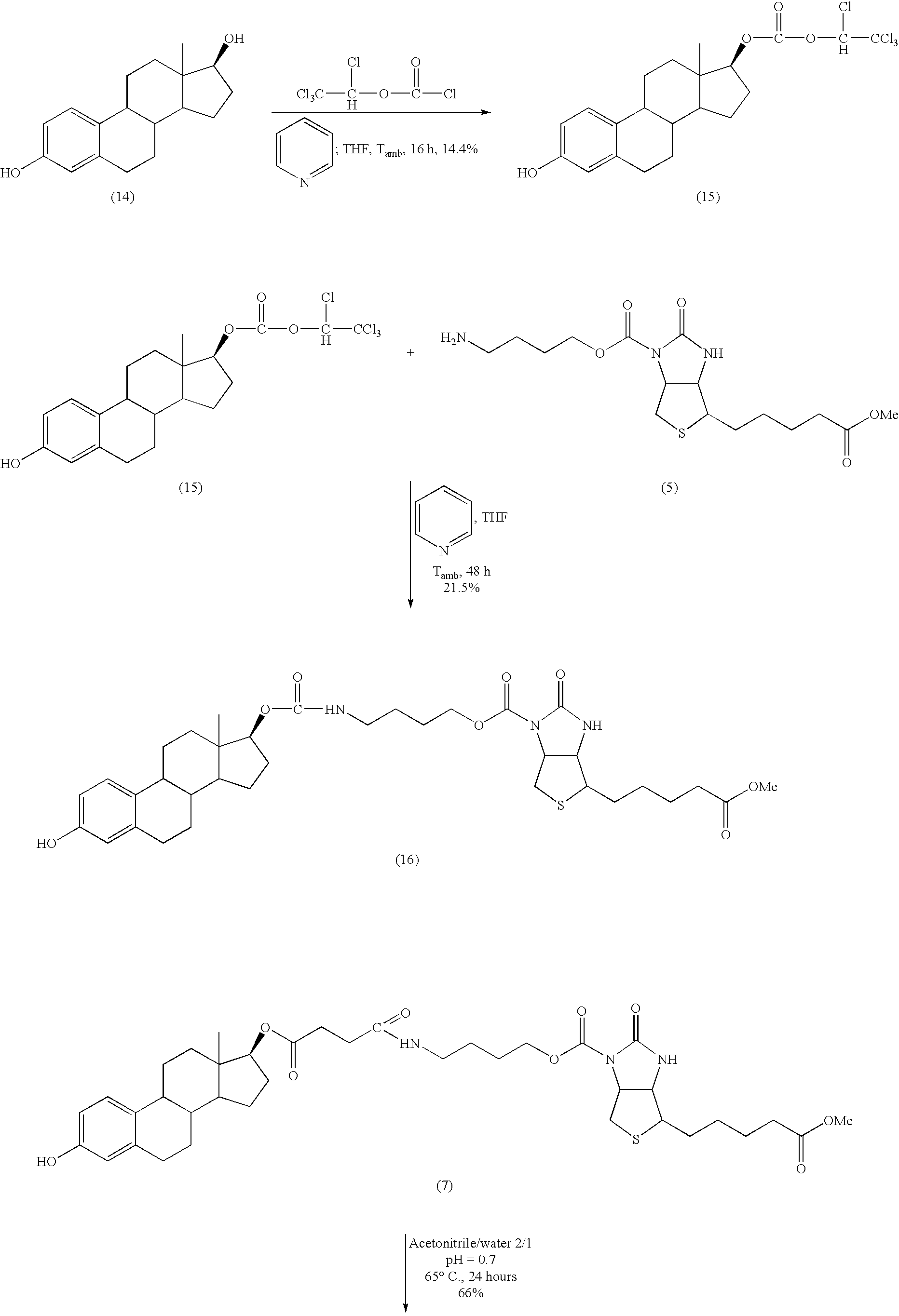

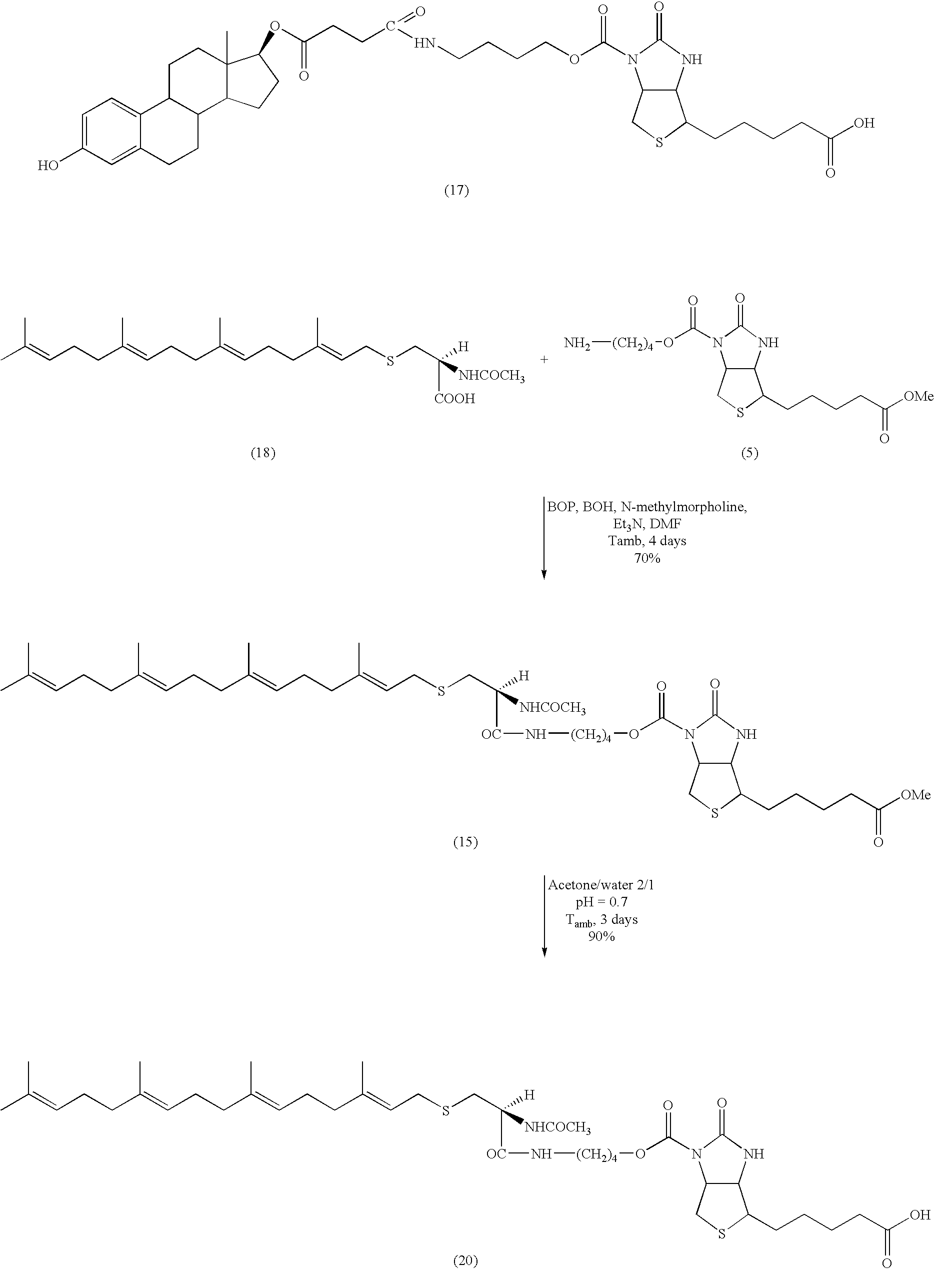

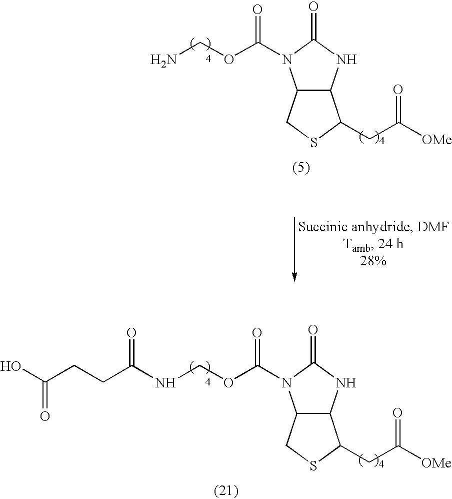

II) Cloning of the Genes of Fusion of BCCP and the Revealing Proteins

The BCCP gene is amplified by PCR (Polymerase Chain Reaction) from Escherichia coli genomic DNA.

“GAL4 DNA-binding domain—BCCP” fusion: the BCCP gene is inserted (NcoI/BamHI sites) into the eukaryotic expression vector pAS1-CYH2, as a fusion with the gene corresponding to the GAL4 DNA-binding domain (amino acids 1 to 147 of GAL4). The fusion gene is under the control of a constitutive promoter.

“LexA DNA-binding domain—BCCP” fusion: the BCCP gene is inserted (ApaI/BamHI sites) into the prokaryotic expression vector pTTQ19CAT, as a fusion with the gene corresponding to the LexA DNA-binding domain (amino acids 1 to 81 of LexA), itself inserted via the SphI/ApaI sites. The fusion gene is under the control of an IPTG-inducible ptac promoter.

The sequences encoding the fusion proteins are indicated below:

GAL4 DNA-binding domain—BCCP:

MKLLSSIEQACDICRLKKLKCSKEKPKCAKSLKNNWECRYSPKTKRSPLTRAHLT
EVESRLERLEQLFLLIFPREDLDMIFKMDSLQDIKALLTGLFVQDNVKDAVTDR
FASVETDMPLTLRQHRISATSSSEES˜INKGQRQLTVSPEFMAYPYDVPDYASLG
GHMDIRKIKKLIELVEESGISELEISEGEESVRISRAAPAASFPVMQQAYAAPMM
QQPAQSNAAAPATVPSMEAPAAAEISGHIVRSPMVGTFYRTPSPDAKAFIEVGQK
VNVGDTLCIVEAMKMMNQIEADKSGTVKAILVESGQPVEFDEPLVVIEZGS

LexA DNA-binding domain—BCCP:

| ACQSVNGQATRGVZSHPZSHQPDRYAADACGNRAAFGVPFPKRGZRTSEGAGTQR | | | | | |
|---|---|---|---|---|---|
| RYZNCFRRITRDSSVAGRGRRVAAGRSWARYSZDZKTDRAGZRIRHLRTGNFZRR | | | | | |
| RVSTHZPCSSCRKFPCDATSLRCTNDAAASSIZRSRSGDRSFHGSASSSGNQWSH | | | | | |
| RTFPDGWYFLPHPKPGRKSVHRSGSESQRGRYPVHRZSHENDEPDRSGQIRYRES | | | | | |
| NSGRKWTTGRIZRAAGRHRVRI | | | | | |
| A = Ala | C = Cys | D = Asp | E = Glu | F = Phe | G = Gly |
| H = His | I = Ile | K = Lys | L = Leu | M = Met | N = Asn |
| P = Pro | Q = Gln | R = Arg | S = Ser | T = Thr | V = Val |
| W = Trp | Y = Tyr | | | | |

Experimental Section—Two-Hybrid Test in Yeast

A human placental cell cDNA library is fused with the gene of the GAL4 activating domain. Fragments of 500 to 1500 base pairs cloned into Pact2 as a fusion with GAL4-ad, marketed by Clontech.

The two-hybrid test is developed according to the method of Chang Bai and Stephen J. Elledge, Methods in Enzymology, 283, pp. 141-156, 1997.

The plasmids used are eukaryotic expression vectors: pAS1-CYH2-BCCP which contains the Biotin Carboxylase Carrier Protein (BCCP) gene as a fusion with the gene of the GAL4 DNA-binding domain, and the gene for auxotrophy with respect to tryptophan; pACTII-RhoGDI3 which contains the RhoGDI3 gene as a fusion with the gene of the GAL4 activating domain, and the gene for auxotrophy with respect to leucine.

The yeast strain Y190 (GAL4 operator-β-galactosidase gene, deficient in tryptophan and leucine) is transformed with pAS1-CYH2-BCCP and pACTII-RhoGDI3, plated out onto a dish containing a medium deficient in Leu and Trp: SC Leu-Trp-, and incubated for 3 days at 30° C. The transformed yeast are cultured on SC Leu-Trp-medium deficient in biotin, for 24 h at 30° C., and then on SC Leu-Trp-medium deficient in biotin and supplemented with 1 nM biotin-ligand for 6 h at 30° C. A β-galactosidase activity assay is carried out.

IV. Industrial Applications

The biotin derivatives in accordance with the invention find a particularly advantageous application in the following fields, in a nonlimiting manner:

Application A: Production of fluorescent bifunctional biotins which can be used as protein, RNA and DNA labels, and which can be marketed in the form of a specific fluorescent labeling kit.

Industrial applications: These products are directed toward the biochemical and molecular biological research market. Their potential for use is varied, the main advantages being as follows:

They allow an improvement in biotin-avidin technology, which currently has many fields of application in fundamental and applied research and uses nonfluorescent biotins.

The fluorescent biotin molecules can be used as a replacement for radioactive molecules, with an obvious benefit for user safety and environmental problems. In addition, their use would make it possible to avoid the long and expensive processes of storage and decontamination of radioactive waste.

It is possible to envision new applications, such as the labeling of proteins in vivo, and the simultaneous use of biotins coupled to various fluorescent groups, which would make it possible to obtain, at the same time, several pieces of information regarding the molecular mechanisms of the cell.

Application B: Development of in vivo hormone assays (making it possible to assay an overall activity) which can be marketed in the form of ready-to-use kits.

Industrial applications: The applications are multiple:

In medical test laboratories: for controlling the hormone levels in individuals. Specifically, hormone levels are responsible for the individual's physiological equilibrium, and it is important to be able to assay them at various occasions, puberty, menopause, for the treatment of hormone-dependent cancers for optimal use of estrogen replacement therapy, for the re-balancing of hormones in sports people, etc.

In quality control among veterinarians for detecting the hormone level of meats (veal, chicken, etc.).

For detecting doping products in sports people.

In the pharmaceutical industry for defining new hormone agonists and antagonists.

Application C: Coupling molecules of interest onto biotin and association with permeating peptides for uses as biovectors as a function of the peptide coupled, possibility of transporting the molecule to the cytoplasm or to the nucleus of cells.

Industrial applications: The main market is, in this case, that of gene therapy, for transporting prodrugs or adenoviruses into the cell.

Application D: Identification of primary protein targets (search for targets of novel molecules) and secondary protein targets (determination of side effects) of small nonpeptide molecules in Escherichia coli.

Industrial applications: They lie in the research and development of medicinal products, at two distinct levels:

Identifying the primary and secondary targets of molecules. This is necessary for placing novel medicinal products on the market.

Improving the efficacy of medicinal products.

Application E: Selection of catalytic antibodies from an antibody library (“phage display” method).

Industrial applications: They are multiple. Mention may, for example, be made of;

in medicine, for the conversion of prodrugs at precise sites in the cell or for correcting defective functions of cells.

In the chemical industry: for recycling polymers for example.

V. Pharmaceutical Application

The compounds according to the invention can be administered in pharmaceutical preparations at doses of between 0.1 mg and 10 mg per day and per kilo of weight of the individual.

1. A biotin derivative of general formula (I) below 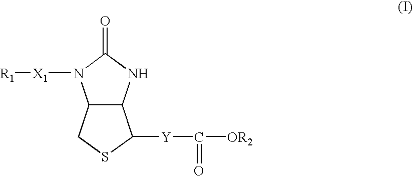
in which
R₁ represents a given compound,
X₁ represents a carbonaceous chain, preferably of 1 to 10 carbon atoms, which may or may not be substituted, comprising, where appropriate, one or more carbonyl or oxycarbonyl groups and/or an —NH— group, in particular a chain of formula —HN(CH₂)ₙ—O—CO— in which n is an integer from 1 to 5, and comprising, where appropriate, one or more ether, amide and/or amine functions,
Y represents a hydrocarbon-based chain of 1 to 5 carbon atoms, which may or may not be substituted,
R₂ represents a hydrogen atom or a protective group, in particular R₂ represents an alkyl group of 1 to 5 carbon atoms.

2. The biotin derivative of formula (I) as claimed in claim 2, chosen from the following:
the compounds of formula (Ia) below 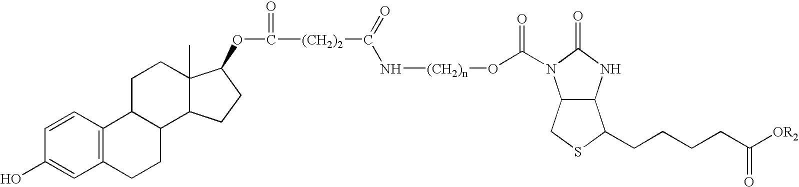
in which:
R₁ represents a radical derived from estradiol hemisuccinate of formula below 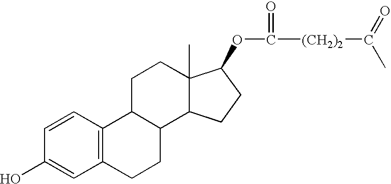
X₁ represents a chain of formula HN—(CH₂)ₙ—O—CO in which n=2 or 4, Y represents a hydrocarbon-based chain of 4 carbon atoms, and R₂ represents —H or CH₃,
the compounds of formula (Ib) below 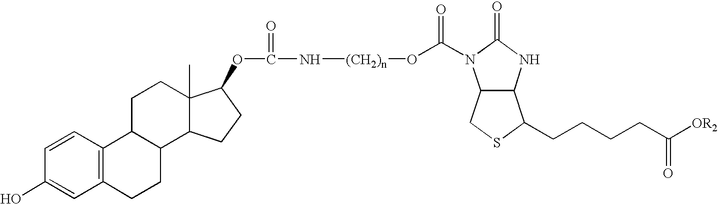
in which
R₁ represents a radical derived from estradiol of formula below 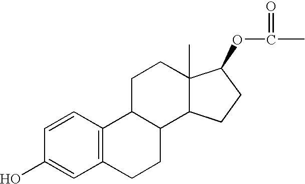
X₁ represents a chain of formula HN—(CH₂)ₙ—O—CO in which n=2 or 4, Y represents a hydrocarbon-based chain of 4 carbon atoms, and R₂ represents —H or —CH₃,
the compounds of formula (Ic) below 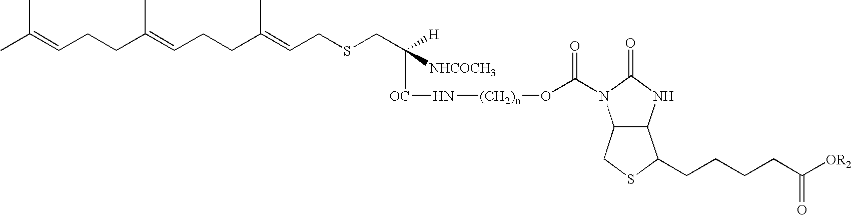
in which
R₁ represents a radical derived from N-acetyl-S-farnesylcysteine of formula below 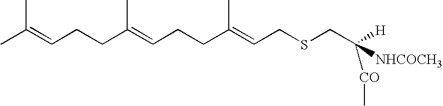
X₁ represents a chain of formula —HN—(CH₂)ₙ—O—CO— in which n=2 or 4, Y represents a hydrocarbon-based chain of 4 carbon atoms, and R₂ represents —H or —CH₃,
the compounds of formula (Id) below 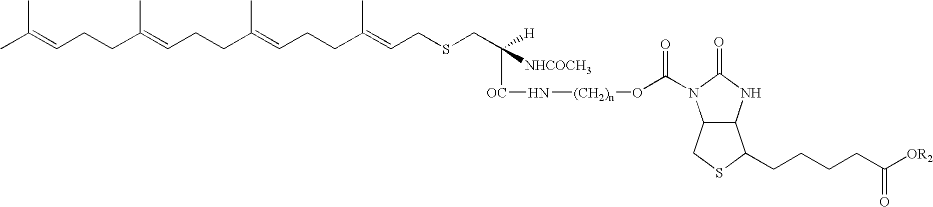
in which
R₁ represents a radical derived from N-acetyl-S-geranylgeranylcysteine of formula below 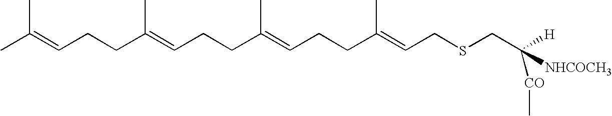
X₁ represents a chain of formula —HN—(CH₂)ₙ—O—CO— in which n=2 or 4, Y represents a hydrocarbon-based chain of 4 carbon atoms, and R₂ represents —H or —CH₃,
the compounds of formula (Ie) below 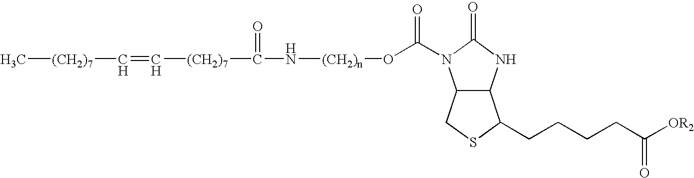
R₁ represents a radical derived from oleic acid of formula below
H₃C—(CH₂)₇—CH═CH—(CH₂)₇—CO—
X₁ represents a chain of formula HN—(CH₂)ₙ—O—CO— in which n=2 or 4, Y represents a hydrocarbon-based chain of 4 carbon atoms, and R₂ represents —H or —CH₃.

3. A biotin derivative of general formula (II) below 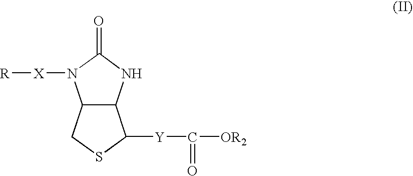
in which
R represents a function —COOH, —NH₂ or —N₃, or a halogen atom,
X represents a carbonaceous chain, preferably of 1 to 10 carbon atoms, which may or may not be substituted, comprising, where appropriate, one or more carbonyl or oxycarbonyl groups, in particular a chain of formula —(CH₂)ₙ—O—CO in which n is an integer of 1 to 5,
Y represents a hydrocarbon-based chain of 1 to 5 carbon atoms, which may or may not be substituted,
R₂ represents a hydrogen atom or a protective group, in particular R₂ represents an alkyl group of 1 to 5 carbon atoms.

4. The biotin derivative of formula (II) as claimed in claim 3, of one of the formulae below

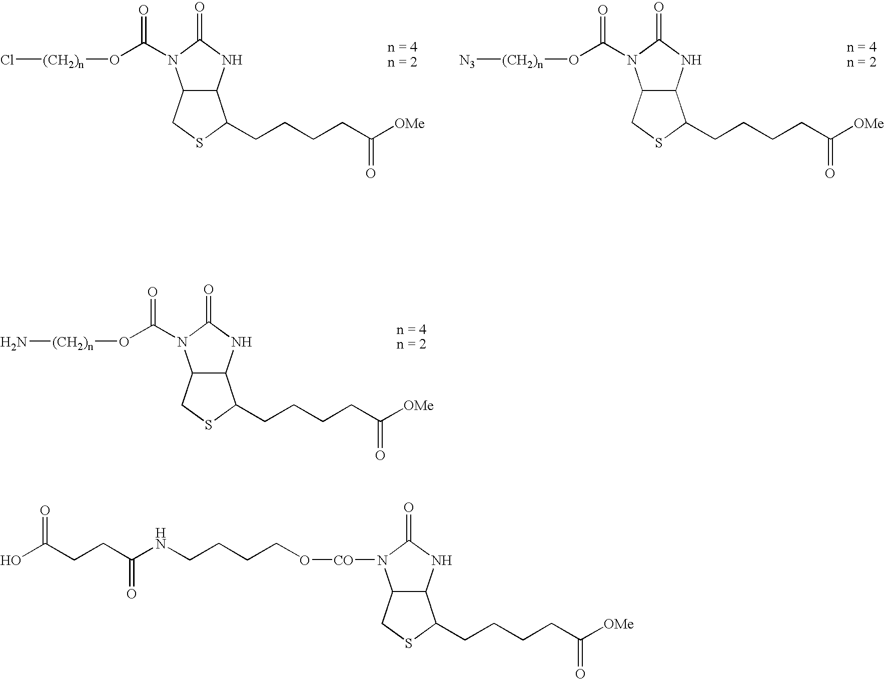

5. A method for preparing biotin derivatives as claimed in one of claims 1 to 4, comprising the following steps:
treating a biotin derivative of formula below 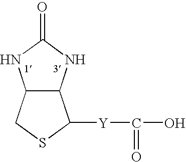
in which Y is as defined in claim 1 or 3, with a compound of formula R₂—OH, in which R₂ represents a protective group, in the presence of an ion exchange resin, advantageously at approximately 70° C. for approximately 7 hours, which produces the compound of formula below 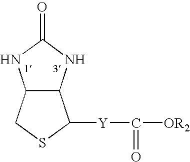
in which Y and R₂ are as defined above,
bringing the ester obtained in the preceding step into contact with a compound of formula R—X—Cl in which R is as defined in claim 3, in particular R represents a halogen atom such as Cl or I, and X is as defined in claim 3, in particular X represents a chain of formula —(CH₂)ₙ—O—CO— in which n represents 2 or 4, advantageously at approximately 65° C. for 4 days, which leads to the production of the compound of formula (IIa) below 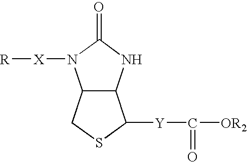
in which X, R, Y and R₂ are as defined above,
where appropriate, treating the compound of abovementioned formula (IIa) in which R represents a halogen atom, with NaN₃/NaI, advantageously at approximately 60° C. for 4 days, which leads to the production of the compound of formula (IIb), namely a compound of formula (II) in which R represents N₃, and X, Y and R₂ are as defined above
where appropriate, hydrogenating the compound of abovementioned formula (IIb), which leads to the production of the compound of formula (IIc), namely a compound of formula (II) in which R represents NH₂, and X, Y and R₂ are as defined above,
where appropriate, coupling the compounds of abovementioned formulae (IIa), (IIb) and (IIc) with a given compound, using a coupling reagent such as BOP/BOH in the presence of triethyl-amine, which leads to the production of compounds of formula (I) in which R₁, X₁ and Y are as defined in claim 1, and R₂ is as defined above,
where appropriate, deprotecting the compounds of abovementioned formulae (I) and (II), in particular:
by treatment of said compounds with an esterase, such as pig liver esterase, at ambient temperature for approximately 10 days, or
by acid catalysis of said compounds, in particular by treatment with sulfuric acid at 65° C. for 24 hours, which leads to the production of the compounds of abovementioned formulae (I) and (II) in which R₂═H.

6. The use of biotin derivatives as claimed in claim 1 or 2, as vectors for introducing said given compound into target cells, in the context of implementing a method for detecting proteins or other nonprotein molecules capable of interacting with said given compound, or in the context of preparing medicinal products for introducing a given compound of therapeutic interest into cells of the organism.

7. A method for detecting proteins capable of interacting with the radical R₁ of a biotin derivative as claimed in claim 1 or 2, characterized in that it comprises:
bringing a biotin derivative as claimed in claim 1 or 2 into contact with host cells transformed so as to contain:
a DNA sequence encoding a first fusion protein comprising BCCP and a first revealing protein, and
a DNA sequence encoding a second fusion protein comprising a protein capable of interacting with the abovementioned radical R₁ and a second revealing protein,
the two revealing proteins being chosen in such a way that the bringing together thereof, subsequent to an interaction between the protein capable of interacting with the radical R₁ in said second fusion protein, and the radical R₁ of said biotin derivative, engenders a reaction process the result of which can be detected, this bringing into contact being carried out for a period of time sufficient to allow the transport of said biotin derivative into said host cells, and the binding of said biotin derivative to the BCCP of said first fusion protein,
possibly detecting the result of the reaction process due to the bringing together of the two revealing proteins, which reflects an interaction between said protein capable of interacting with the radical R₁, and said radical R₁.

8. A method for detecting protein or nonprotein molecules, tested for their ability to interact with the radical R₁ of a biotin derivative as claimed in claim 1 or 2, characterized in that it comprises
bringing
a first biotin derivative as claimed in claim 1 or 2, in which R₁ represents the given compound with which the tested molecule is capable of interacting, and
a second biotin derivative as claimed in claim 1 or 2, of formula (I) in which R represents the tested molecule, into contact with host cells transformed so as to contain:
a DNA sequence encoding a first fusion protein comprising BCCP and a first revealing protein, and
a DNA sequence encoding a second fusion protein comprising BCCP and a second revealing protein,
the two revealing proteins being chosen in such a way that the bringing together thereof, subsequent to an interaction between said tested molecule of the second biotin derivative and said radical R₁ of the first biotin derivative, engenders a reaction process the result of which can be detected,
this bringing into contact being carried out for a period of time sufficient to allow the transport of the abovementioned first and second biotin derivatives into said host cells, and the binding of said biotin derivatives to the BCCP of said fusion proteins,
possibly detecting the result of the reaction process due to the bringing together of the two revealing proteins, which reflects an interaction between said tested molecule of the second biotin derivative and said radical R₁ of the first biotin derivative.

9. A pharmaceutical composition, characterized in that it comprises one or more biotin derivatives as claimed in claim 1 or 2, of formula (I) in which R₁ represents a compound of therapeutic interest, in combination with a pharmaceutically acceptable vehicle.

10. A nucleotide sequence encoding a fusion protein comprising BCCP and a revealing protein, for implementing a method as claimed in claim 7 or 8, chosen from
the nucleotide sequence containing the DNA sequence encoding the GAL4 DNA-binding domain, said DNA sequence being linked to the DNA sequence encoding BCCP,
the nucleotide sequence containing the DNA sequence encoding the GAL4 activating domain, said DNA sequence being linked to the DNA sequence encoding BCCP,
the nucleotide sequence containing the DNA sequence encoding the LexA DNA-binding domain, said DNA sequence being linked to the DNA sequence encoding BCCP,
the nucleotide sequence containing the DNA sequence encoding the LexA dimerization domain, said DNA sequence being linked to the DNA sequence encoding BCCP,
the nucleotide sequence containing the DNA sequence encoding GFP, said DNA sequence being linked to the DNA sequence encoding BCCP,
the nucleotide sequence containing the DNA sequence encoding BFP, said DNA sequence being linked to the DNA sequence encoding BCCP.

11. A vector, in particular a plasmid, containing a nucleotide sequence defined in claim 10.

12. A host cell transformed with a vector defined in claim 11.

13. A fusion protein comprising BCCP fused to a revealing protein as described in claim 10, in particular
the protein from fusion between the GAL4 DNA-binding domain and BCCP,
the protein from fusion between the GAL4 activating domain and BCCP,
the protein from fusion between the LexA DNA-binding domain and BCCP,
the protein from fusion between the LexA dimerization domain and BCCP,
the protein from fusion between GFP and BCCP,
the protein from fusion between BFP and BCCP.

14. A set or kit for implementing a detection method as claimed in claim 7 or 8, characterized in that it comprises:
transformed host cells as claimed in claim 12, and/or
biotin derivatives as claimed in claim 3 or 4.
